(19) United States
(12) Patent Application Publication
(10) Pub. No.: US 2010/0216761 A1
(43) Pub. Date: Aug. 26, 2010

(54) NOVEL AZETIDINONES USEFUL AS INHIBITORS OF ELASTASE

(76) Inventors: James B. Doherty, Montebello, NY (US); Paul E. Finke, Milltown, NJ (US); Richard A. Mumford, Red Bank, NJ (US); Kelly M. Treonze, Hillsborough, NJ (US)

Correspondence Address: MERCK, P O BOX 2000, RAHWAY, NJ, 07065-0907, US

(21) Appl. No.: 12/680,289

(22) PCT Filed: Oct. 1, 2008
(86) PCT No.: PCT/US08/11343

§ 371 Date: Mar. 26, 2010

Related U.S. Application Data
(60) Provisional application No. 60/997,903, filed on Oct. 5, 2007.

(51) Int. Cl. A61K 31/397; C07D 205/04; A61P 11/00; A61P 9/00; A61P 19/02; A61P 19/10; A61P 17/06; A61P 31/00

(52) U.S. Cl. 514/210.02; 540/360

(57) ABSTRACT

The invention is directed to novel azetidinones selected from 2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidin ecarboxamide, 2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and analogs thereof, and pharmaceutically acceptable salts thereof, and their use in the treatment of diseases associated with an excess of elastase, including emphysema, bronchial inflammation, chronic bronchitis, cystic fibrosis, acute respiratory distress syndrome, rheumatoid arthritis, osteoarthritis; glomerulonephritis, spondylitis, lupus, psoriasis, atherosclerosis, sepsis, septicemia, shock, myocardial infarction, reperfusion injury, and periodontitis.

### BACKGROUND OF THE INVENTION

Proteases from granulocytes and macrophages have been reported to be responsible for the chronic tissue destruction mechanisms associated with inflammation, including rheumatoid arthritis and emphysema. Accordingly, specific and selective inhibitors of these proteases are candidates for potent anti-inflammatory agents useful in the treatment of inflammatory conditions resulting in connective tissue destruction, e.g. rheumatoid arthritis, emphysema, bronchial inflammation, chronic bronchitis, glomerulonephritis, osteoarthritis, spondylitis, lupus, psoriasis, atherosclerosis, sepsis, septicemia, shock, myocardial infarction, reperfusion injury, periodontitis, cystic fibrosis and acute respiratory distress syndrome.

The role of proteases from granulocytes, leukocytes or macrophages are related to a rapid series of events which occurs during the progression of an inflammatory condition:

(1) There is a rapid production of prostaglandins (PG) and related compounds synthesized from arachidonic acid. This PG synthesis has been shown to be inhibited by aspirin-related nonsteroidal anti-inflammatory agents including indomethacin and phenylbutazone. There is some evidence that protease inhibitors prevent PG production;

(2) There is also a change in vascular permeability which causes a leakage of fluid into the inflamed site and the resulting edema is generally used as a marker for measuring the degree of inflammation. This process has been found to be induced by the proteolytic or peptide cleaving activity of proteases, especially those contained in the granulocyte, and thereby can be inhibited by various synthetic protease inhibitors, for example, N-acyl benzisothiazolones and the respective 1,1-dioxides. Morris Zimmerman et al., J. Biol. Chem., 255, 9848 (1980); and

(3) There is an appearance and/or presence of lymphoid cells, especially macrophages and polymorphonuclear leukocytes (PMN). It has been known that a variety of proteases are released from the macrophages and PMN, further indicating that the proteases do play an important role in inflammation.

In general, proteases are an important family of enzymes within the peptide bond cleaving enzymes whose members are essential to a variety of normal biological activities, such as digestion, formation and dissolution of blood clots, the formation of active forms of hormones, the immune reaction to foreign cells and organisms, etc., and in pathological conditions such as the degradation of structural proteins at the articular cartilage/pannus junction in rheumatoid arthritis etc.

Elastase is one of the proteases. It is an enzyme capable of hydrolyzing the connective tissue component elastin, a property not contained by the bulk of the proteases present in mammals. It acts on a protein's nonterminal bonds which are adjacent to an aliphatic amino acid. Neutrophil elastase is of particular interest because it has the broadest spectrum of activity against natural connective tissue substrates. In particular, the elastase of the granulocyte is important because, as described above, granulocytes participate in acute inflammation and in acute exacerbation of chronic forms of inflammation which characterize many clinically important inflammatory diseases.

Proteases may be inactivated by inhibitors which block the active site of the enzyme by binding tightly thereto. Naturally occurring protease inhibitors form part of the control or defense mechanisms that are crucial to the well-being of an organism. Without these control mechanisms, the proteases would destroy any protein within reach. The naturally occurring enzyme inhibitors have been shown to have appropriate configurations which allow them to bind tightly to the enzyme. This configuration is part of the reason that inhibitors bind to the enzyme so tightly (see Stroud, “A Family of Protein-Cutting Proteins” Sci. Am. July 1974, pp. 74-88). For example, one of the natural inhibitors, alpha.sub.1-Antitrypsin, is a glycoprotein contained in human serum that has a wide inhibitory spectrum covering, among other enzymes, elastase both from the pancreas and the PMN. This inhibitor is hydrolyzed by the proteases to form a stable acyl enzyme in which the active site is no longer available. Marked reduction in serum α₁-antitrypsin, either genetic or due to oxidants, has been associated with pulmonary emphysema which is a disease characterized by a progressive loss of lung elasticity and resulting respiratory difficulty. It has been reported that this loss of lung elasticity is caused by the progressive, uncontrolled proteolysis or destruction of the structure of lung tissue by proteases such as elastase released from leukocytes. J. C. Powers, TIBS, 211 (1976).

### SUMMARY OF THE INVENTION

The invention is directed to novel azetidinones selected from 2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide, 2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and analogs thereof, and pharmaceutically acceptable salts thereof, and their use in the treatment of diseases associated with an excess of elastase, including emphysema, bronchial inflammation, chronic bronchitis, cystic fibrosis, acute respiratory distress syndrome, rheumatoid arthritis, osteoarthritis; glomerulonephritis, spondylitis, lupus, psoriasis, atherosclerosis, sepsis, septicemia, shock, myocardial infarction, reperfusion injury, and periodontitis.

### DETAILED DESCRIPTION OF THE INVENTION

In one aspect the invention is directed to a compound selected from the group consisting of
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
   or a pharmaceutically acceptable salt thereof.

Within this aspect there is a compound

2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide, or a pharmaceutically acceptable salt thereof.

Within this aspect there is a compound

2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide, or a pharmaceutically acceptable salt thereof.

In another aspect the invention is directed to a pharmaceutical composition comprising a compound selected from
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
   or a pharmaceutically acceptable salt thereof, and a pharmaceutically acceptable carrier.

In another aspect the invention is directed to a method of treating an elastase mediated disease selected from the group consisting of emphysema, bronchial inflammation, chronic bronchitis, cystic fibrosis, and acute respiratory distress syndrome in a patient having said disease comprising the administration of a non-toxic therapeutically effective amount of a compound selected from:
2-(S)-[4-(((2-(Dimethylamino)ethyl)ethylamino)carbonyl)phenoxy]-3,3-di ethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
   or a pharmaceutically acceptable salt thereof.

In another aspect the invention is directed to a method of treating an elastase mediated disease selected from the group consisting of rheumatoid arthritis and osteoarthritis in a patient having said disease comprising the administration of a non-toxic therapeutically effective amount of a compound selected from:
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methyl amino)carbonyl)phenoxy]-3,3-di ethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
   or a pharmaceutically acceptable salt thereof.
   salt thereof.

In another aspect the invention is directed to a method of treating an elastase mediated disease selected from glomerulonephritis, spondylitis, lupus, psoriasis, atherosclerosis, sepsis, septicemia, shock, myocardial infarction, reperfusion injury, and periodontitis in a patient in need of such treatment comprising the administration therapeutically effective amount of a compound selected from:
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
   or a pharmaceutically acceptable salt thereof.

In another aspect the invention is directed to a method of treating congenital neutropenia, especially severe congenital neutropenia comprising the administration therapeutically effective amount of a compound of the invention.

In another aspect the invention is directed to a method of treating idiopathic mylofibrosis, polycythemia vera, essential thrombocytopenia, aortic aneurism, advanced coronary artery disease and pulmonary hypertension.

In another aspect the present invention is directed to the treatment of leukemia, such as nonlymphoblastic leukemias, acute myelogenous leukemia (FAB M1 and FAB M2), acute promyelocytic leukemia (FAB M3), acute myelomonocytic leukemia (FAB M4), acute monocytic leukemia (FAB M5), erythroleukemia, chronic myelogenous leukemia, chronic myelomonocytic leukemia, chronic monocytic leukemia and conditions associated with leukemia involving activity of PMN neutral proteases e.g. disseminated intravascular coagulation with compounds of the invention.

Treatment of leukemia cells comprises: administration of a therapeutically effective amount of a compound of the invention which administration results in the inhibition of proteinase 3/myeloblastin, inhibition of elastase, inhibition of proliferation of the leukemia cells, induction of differentiation of the leukemia cells, and remission of the disease state.

In one alternative embodiment the invention concerns a method of treating leukemia comprising:

administration to a patient in need of such treatment of a therapeutically effective amount of compound of the invention.

In another aspect the invention is directed to a method of inhibiting proteinase 3/myeloblastin, comprising:

administration to a patient in need of such inhibition of a therapeutically effective amount of compound of the invention.

In another aspect the invention is directed to a method of inhibiting proteinase 3/myeloblastin and elastase, comprising:

administration to a patient in need of such inhibition of a therapeutically effective amount of compound of the invention or a pharmaceutically acceptable salt thereof as defined above.

In another aspect the invention is directed to a method of inducing cellular differentiation in leukemia cells comprising:

administration to a patient in need of such inhibition of a therapeutically effective amount of compound of the invention or a pharmaceutically acceptable salt thereof as defined above.

Each of the methods relating to PR3 or cancer also concerns co-administration of a compound of the invention as defined above, with an agent or agents known in the art for treatment of leukemia, including, but not limited to epsilon-aminocaproic acid, heparin, trasylol (aprotinin); prednisolone; cytosine arabinoside; .beta.-mercaptopurine; cytarabine; an anthracycline (see Young et. al. (1981) N. Engl. J. Med. 305:139) such as daunorubicin, doxorubicin and epidoxorubicin; Vitamin A derivatives including retinoids and all-trans-retinoic acid (See Ellison R. R. et. al. (1968) Blood 32:507, Arabinosyl Cytosine: A useful agent in the treatment of leukemia in adults; Cytarabine: Therapeutic new dimensions, Semin. Oncol. 12:1 (1985, supp 3); Weinstein H. J. et. al. (1983) Blood 62:315, Chemotherapy for acute myelogenous leukemia in children and adults results in an enhanced therapeutic response.

Accordingly, in another aspect the invention is directed to a pharmaceutical composition comprising:

a pharmaceutical carrier, a therapeutically effective amount of compound selected from the group consisting of epsilon-aminocaproic acid, heparin, trasylol, prednisolone, cytosine arabinoside, .beta.-mercaptopurine, cytarabine, an anthracycline and a vitamin A derivative; and a therapeutically effective amount of compound of the invention.

In another aspect the invention is directed to a method of treating leukemia comprising:

co-administration to a patient in need of such treatment of a therapeutically effective amount of compound selected from the group consisting of epsilon-aminocaproic acid, heparin, trasylol, prednisolone, cytosine arabinoside, b-mercaptopurine, cytarabine, an anthracycline, and a vitamin A derivative; and a therapeutically effective amount of compound of the invention.

In another aspect the invention is directed to a method of inhibiting proteinase 3/myeloblastin, comprising:

co-administration to a patient in need of such inhibition of a therapeutically effective amount of compound selected from the group consisting of epsilon-aminocaproic acid, heparin, trasylol, prednisolone, cytosine arabinoside, .beta.-mercaptopurine, cytarabine, an anthracycline, and a vitamin A derivative; and a therapeutically effective amount of compound of the invention.

In another aspect the invention is directed to a method of inhibiting proteinase 3/myeloblastin and elastase, comprising:

administration to a patient in need of such inhibition of a therapeutically effective amount of compound selected from the group consisting of epsilon-aminocaproic acid, heparin, trasylol, prednisolone, cytosine arabinoside, .beta.-mercaptopurine, cytarabine, an anthracycline, and a vitamin A derivative; and a therapeutically effective amount of compound of the invention.

In another aspect the invention is directed to a method of inducing cell differentiation in leukemia cells comprising:

administration to a patient in need of such inducing of a therapeutically effective amount of compound selected from the group consisting of epsilon-amino-caproic acid, heparin, trasylol, prednisolone, cytosine arabinoside, .beta.-mercaptopurine, cytarabine, an anthracycline and a vitamin A derivative; and a therapeutically effective amount of compound of the invention.

In another aspect the compounds of the invention may also be used in the treatment of diseases associated with over-expression of cDNAa, such as those pulmonary diseases with abnormal, viscous, or inspissated purulent secretions. Such conditions are found in acute or chronic bronchopulmonary disease including infectious pneumonia, bronchitis or tracheobronchitis, bronchiectasis, cystic fibrosis, asthma, tuberculosis or fungal infections. Utility is also found in atelactasis due to tracheal or bronchial impaction and complications of tracheostomy.

In addition, the instant compounds can be co-administered with cDNAase which also finds utility in these pulmonary diseases, and which is described in WO 90/07572.

The following examples illustrate the preparation of the compounds of the invention. Where appropriate, compounds may be produced and used in the form of pharmaceutically acceptable salts. For example, the basic compounds may be used in the form of a hydrochloride or mesylate or other acceptable salt.

### Intermediate 1

### Method A

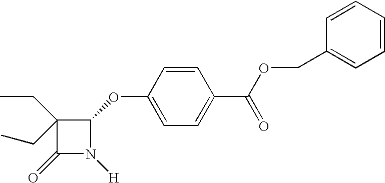

### (R,S)-2-[4-(Benzyloxcarbonyl)phenoxy]-3,3-diethylazetidin-4-one

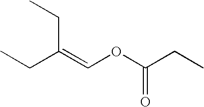

### Step A: Preparation of 1-propionyloxy-2-ethyl-1-butene

A reaction vessel was charged sequentially with Et₃N (192 mL), propionic anhydride (217 mL), dimethylaminopyridine (DMAP, 1.4 gm) and 2-ethylbutyraldehyde (113 mL). The mixture was stirred and heated under gentle reflux (120°-130° C.) for 5 hr under a nitrogen atmosphere. The reaction was then cooled to 50° C. and water (0.5 L) was added slowly (temperature rises to 80° C.). On complete addition, the mixture was heated at reflux for 45 min and then cooled to room temperature before hexanes (1 L) was added. The aqueous layer was separated and re-extracted with hexanes (0.5 L) and the combined organic layers were washed with saturated NaHCO₃ and brine before being concentrated in vacuo at 840° C. The residue so obtained was fractionally distilled (b.p. 75°-85° C., 30-40 mm Hg) to give the title product (145 gm) as a clear liquid.

### Step B: Preparation of 2-propionyloxy-3,3-diethylazetindin-4-one

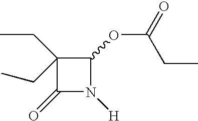

To a cooled solution of 1-propionyloxy-2-ethyl-1-butene (140 gm) (from Step A) in nitromethane (75 mL) under a nitrogen atmosphere was added chlorosulfonyl isocyanate (120 mL) over 30 min, maintaining the temperature below 6° C. with an ice/methanol bath. On complete addition, the yellow solution was re-cooled to below 0° C. and stored under a nitrogen atmosphere at 0° C. for 60 hours. The reaction mixture was then diluted with ether (1 L) and then added slowly over 30 min to a mixture of ice water (2 L), Na₂SO₃ (200 gm), NaHCO₃ (360 gm) and ether (1 L), maintaining the temperature below 5° C. throughout the addition. An additional 0.5 L of ether was used for washing-in. The reaction was then allowed to rise to 15° C. over 2 hr, after which time gas evolution had ceased. The lower layer was separated and further extracted twice with ether. The combined organic extracts were washed with NaHCO₃ solution and brine, dried over Na₂SO₄, filtered and evaporated to dryness to give the title product (150 gm) as a thick, red oil suitable for use in the next step (solidifies on storage at −10° C.).

### Step C: Preparation of (S), (R) and (R,S)-2-[4-(benzyloxycarbonyl)phenoxy]-3,3-diethylazetidin-4-one

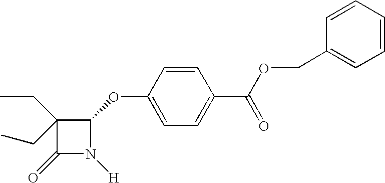

A mechanically stirred solution of 2-propionyloxy-3,3-diethylazetindin-4-one

(100 gm) (prepared in Step B), benzyl 4-hydroxybenzoate (82.4 gm), (+)-cinchonin (14 gm) and powdered anhydrous Na₂CO₃ (50 gm) in toluene (1200 mL) under nitrogen was heated to 45° C. for 96 hr, then at 55° C. for another 96 hr. After cooling, the solids were removed by filtration and ether (1000 ml) was added. The solution was washed successively with water, sat. NaHCO₃ solution and brine. The solution was then dried over Na₂SO₄, filtered and evaporated to dryness to give a clear, yellow oil. Crystallization from EtOAc/hexanes (250 mL/1000 mL) afforded essentially pure title (S) compound (21 gm) which contained only a trace of the (R) enantiomer. The mother liquor was concentrated and purified by flash chromatography (silica gel, 25% EtOAc in hexanes) to give essentially racemic title compound. Chiral separation on an OD Chiracel column afforded additional pure (S) enantiomer and pure (R) enantiomer.

NMR (CDCl₃): δ1.08 (t, 3H), 1.12 (t, 3H), 1.64-2.14 (m, 4H), 3.48 (s, 2H), 5.18 (s, 2H), 5.38 (s, 1H), 6.90 (d, 2H), 7.28 (d, 2H), 7.40 (s, 5H).

### Intermediate 1

### Method B

### Preparation of (R,S)-2-[4-(benzyloxycarbonyl)phenoxy]-3,3-diethylazetidin-4-one

Benzyl 4-hydroxybenzoate is dissolved in DMF and water and milled K₂CO are added. To this mixture at 35° C. is added 2-propionyloxy-3,3-diethylazetindin-4-one (prepared as in Method A, Step B). The resulting mixture is cooled and stirred at 30° C. for 1 hr, followed by stirring at 18° C. for an additional 1 hr before being quenched by the addition of 2N HCl and EtOAc. The layers are separated and the aqueous phase (pH 8.2) was further extracted with EtOAc. The combined organic layers are washed successively with sat. NaHCO₃, water, and brine before being concentrated in vacuo to afford the title compound as an oil which can be further purified as in Method A.

### Intermediate 1

### Method C

### Preparation of (R) and (S)-2-[4-(benzyloxycarbonyl)phenoxy]-3,3-diethylazetidin-4-one

An enzymatic resolution for Intermediate 1 is also known in the literature. See: Cvetovich, Raymond J.; Chartrain, Michel; Hartner, Frederick W., Jr.; Roberge, Christopher; Amato, Joseph S.; Grabowski, Edward J. J. “An Asymmetric Synthesis of L-694,458, a Human Leukocyte Elastase Inhibitor, via Novel Enzyme Resolution of β-Lactam Esters” Journal of Organic Chemistry (1996), 61(19), 6575-6580.

### Intermediate 2

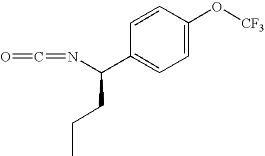

### (R)-α-Allyl-4-(trifluoromethoxy)benzyl isocyanate

### Step A: Preparation of N—[(R)-α-allyl-4-(trifluoromethoxy)benzyl]-(S)-t-butylsulfinamide

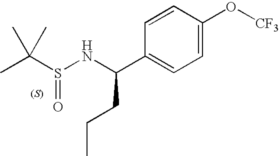

To a solution of 4-(trifluoromethoxy)benzaldehyde (8.5 gm) and (S)-(−)-t-butylsulfinamide (6.0 gm) in DCE (40 mL) under nitrogen in a pressure bottle was added CuSO₄ (12 gm). The reaction was sealed and heated at 75° C. for 18 hr, then stirred at rt for 48 hr. The reaction was diluted with DCM, filtered through celite and concentrated in vacuo. The residue was directly eluted through silica (5% ethyl acetate/hexanes to remove residual aldehyde, then 10-15% ethyl acetate/hexanes to elute the imine intermediate (13.5 gm).

NMR (CDCl₃): δ 1.27 (s, 9H), 7.32 (d, 2H), 7.91 (d, 2H).

The above imine was taken up in THF (250 mL) and cooled in an ice/methanol bath before slow addition of 2N allylmagnesium chloride in THF (34 mL). The reaction was allowed to warm to rt over 18 hr and was then quenched into dilute an aq HCl/ether mixture. The layers were separated and the aq layer was extracted with ether. The combined organic layers were washed with NaHCO₃ solution and brine, dried over Na₂SO₄ and concentrated. The residue was purified by FC with 10-40% ethyl acetate/hexanes to afford the higher, minor (S)-allyl diastereomer and then the major, desired (R)-allyl title compound (15 gm).

Higher Rf, (S)-allyl NMR (CDCl₃): δ 1.23 (s, 9H), 2.54 and 2.73 (tABq, 2H), 3.52 (br s, 1H), 4.47 (dd, 1H), 5.03 (m, 1H), 5.07 (m, 1H), 5.60 (m, 1H), 7.19 (d, 2H), 7.36 (d, 2H).

Lower Rf, (R)-allyl NMR (CDCl₃): δ 1.21 (s, 9H), 2.46 and 2.58 (tABq, 2H), 3.65 (br s, 1H), 4.49 (dd, 1H), 5.18 (m, 1H), 5.21 (m, 1H), 5.72 (m, 1H), 7.19 (d, 2H), 7.35 (d, 2H).

### Step B: Preparation of (R)-α-allyl-4-(trifluoromethoxy)benzylamine hydrochloride

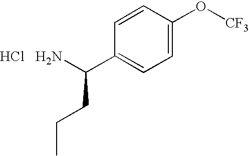

The N—[(R)-α-allyl-4-(trifluoromethoxy)benzyl]-(S)-t-butylsulfinamide from Step A (15 gm) was taken up in ether (200 mL) and 2N HCl in ether (100 mL) was added under nitrogen. The mixture was stirred as the hydrochloride product rapidly precipitated to give a thick slurry. After aging for 2 hr, the volatiles were removed in vacuo and the residue was dried under a stream of nitrogen in a hood for 24 hr to give the title hydrochloride solid (10 gm).

### Step C: Preparation of (R)-α-allyl-4-(trifluoromethoxy)benzyl isocyanate

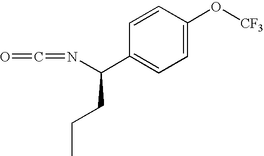

The solid (R)-α-allyl-4-(trifluoromethoxy)benzylamine hydrochloride (10 gm) from Step B was suspended in ethyl acetate (250 mL) and phosgene (20% in toluene, 38 mL) was added. The reaction was heated to 80° C. for 2 hr and then most of the ethyl acetate and toluene was distilled at up to 110° C. oil bath temperature to leave the title intermediate as a concentrated solution in toluene.

### Intermediate 3

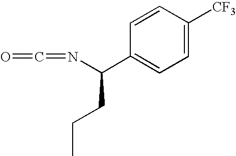

### (R)-α-Allyl-4-(trifluoromethyl)benzyl isocyanate

The title compound was prepared as a toluene solution using the methods used for Intermediate 2 but starting from 4-trifluoromethylbenzaldehyde.

### Example 1

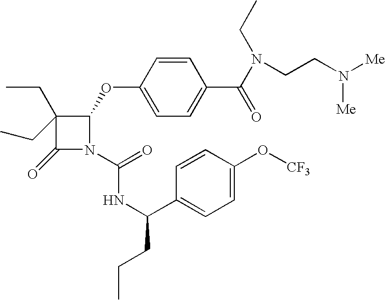

### 2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide

### Step A: Preparation of 2-(S)-[4-(benzyloxycarbonyl)phenoxy]-3,3-diethyl-N-[1-(R)-(4-(trifluoromethoxy)phenyl)but-3-en-yl]-4-oxo-1-azetidinecarboxamide

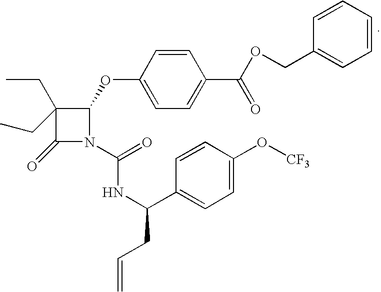

The toluene solution of (R)-α-allyl-4-(trifluoromethoxy)benzyl isocyanate (Intermediate 2 from above) was taken up in DCM (200 mL) and (S)-2-[4-(benzyloxycarbonyl)phenoxy]-3,3-diethyl-azetidin-4-one (Intermediate 1 from above) (10.5 gm), DMAP (0.5 gm) and DIPEA (13.3 mL) were added under nitrogen. The reaction was heated to 45° C. for 21 hr. The mixture was diluted with DCM and poured into water and 18% citric acid (about neutral pH). The aq was re-extracted with DCM and the organic layers were washed with brine, dried over Na₂SO₄ and concentrated in vacuo. The residue was purified in two portions on a Prep 500 instrument (5-10% EtOAc in hexanes) to afford the title 2-(S),N-1-(R) diastereomer as the higher Rf product with only a trace of the lower 2-(S),N-1-(S) diastereomer being produced.

NMR (CDCl₃): δ 0.97 (t, 3H), 1.15 (t, 3H), 1.81 (m, 3H), 1.98 (m, 1H), 2.56 (t, 2H), 4.95 (q, 1H), 5.14 (br s, 1H), 5.18 (br d, 1H), 5.33 (ABq, 2H), 5.63 (m, 1H), 5.69 (s, 1H), 7.01 (d, 1H), 7.17-7.32 (3 br d, 6H), 7.32-7.44 (m, 5H), 8.04 (dt, 2H).

### Step B: Preparation of 2-(S)-(4-(carboxy)phenoxy)-3,3-diethyl-N-[1-(R)-(4-(trifluoromethoxy)phenyl)butyl]-4-oxo-1-azetidinecarboxamide

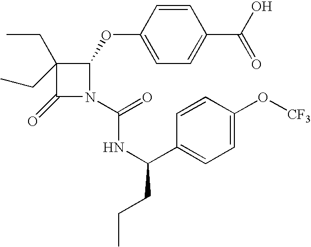

To a solution of 2-(S)-(4-(benzyloxycarbonyl)phenoxy)-3,3-di ethyl-N-(1-(R)-(4-(trifluoromethoxy)phenyl)but-3-en-yl)-4-oxo-1-azetidinecarboxamide (12.5 gm) (from Example 1, Step A) in a solvent mixture of EtOAc (100 mL) and EtOH (50 mL) was added 10% Pd(OH)₂ on carbon (250 mg). The mixture was hydrogenated on a Parr shaker at 40 psi for 2 hr. The catalyst was removed by filtration and the solution was evaporated to dryness to afford the title compound (10.66 gm) as a white foam after evaporation of an aliquot of ether.

NMR (CDCl₃): δ 0.92 (t, 3H), 0.97 (t, 3H), 1.07 (t, 3H), 1.2-1.4 (m, 2H), 1.7-1.9 (2 m, 5H), 1.98 (m, 1H), 4.85 (q, 1H), 5.72 (s, 1H), 6.93 (d, 1H), 7.15-7.33 (3 br d, 6H), 8.04 (dt, 2H).

### Step C: Preparation of 2-(S)-[4-(((2-(dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide

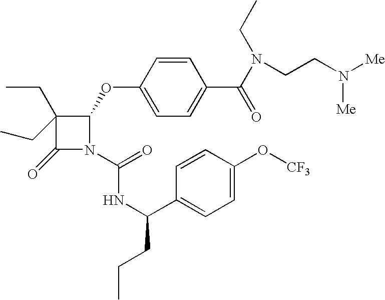

To a solution of 2-(S)-(4-(carboxy)phenoxy)-3,3-diethyl-N-[1-(R)-(4-(trifluoromethoxy)phenyl)butyl]-4-oxo-1-azetidinecarboxamide (10.56 gm) (from Example 1, Step B) in DCM (100 mL) was added DMF (cat, 0.10 mL) and then oxalyl chloride (2.7 mL). The reaction was stirred at rt for 2 hr and was then concentrated in vacuo twice from DCM to give the crude acid chloride as an oil.

The above crude acid chloride was taken up in DCM (150 mL) and cooled in an ice bath before N-ethyl-N′,N′-dimethylethylenediamine (5.8 gm) was added slowly. After 3 hr at rt, the mixture was diluted with DCM and poured into water. The layers were separated and the aq layer was extracted with another two portions of DCM. The organic layers were washed with NaHCO3 solution, then brine, dried over Na₂SO₄ and concentrated in vacuo. The residue was purified by FC eluting first with EtOAc, then 5% MeOH in EtOAC, followed by 1% TEA in 5-10% MeOH in EtOAC. Evaporation and vacuum drying gave the title product as a white solid (11.57 gm).

NMR (CD3CN): δ 0.89 (t, 6H), 1.01 (t, 3H), 1.0-1.2 (br m, 3H), 1.2-1.4 (2 m, 2H), 1.7-1.9 (m, 5H), 1.9-2.3 (m, 7H), 2.3-2.5 (br m, 2H), 3.2-3.4 (2 br m, 4H), 4.80 (q, 1H), 5.73 (s, 1H), 6.92 (d, 1H), 7.18 (dt, 2H), 7.25 (br d, 2H), 7.30 (dt, 2H), 7.39 (dt, 2H).

HPLC/MS (reverse phase, 10-98% CH₃CN/0.05% TFA in water/0.05% TFA over 5.5 min): Rt=3.48 min, m/e=621 (M+1, 100%).

### Example 2

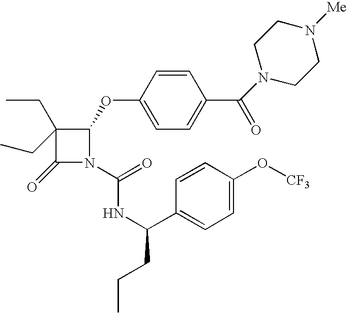

### 2-(S)-[4-((4-Methylpiperidin-1-yl)carbonyl)phenoxy]-3,3-diethyl-N-[1-(R)-(4-(trifluoromethoxy)phenyl)butyl]-4-oxo-1-azetidinecarboxamide

Using the procedure of Example 1, Step C, but using 4-methylpiperidine, 2-(S)-(4-(carboxy)phenoxy)-3,3-diethyl-N-[1-(R)-(4-(trifluoromethoxy)phenyl)butyl]-4-oxo-1-azetidinecarboxamide (50 mg) (prepared as in Example 1, Step B) afforded the title product (38 mg).

HPLC/MS (reverse phase, 10-98% CH₃CN/0.05% TFA in water/0.05% TFA over 5.5 min): Rt=3.63 min, m/e=605 (M+1, 100%).

### Example 3

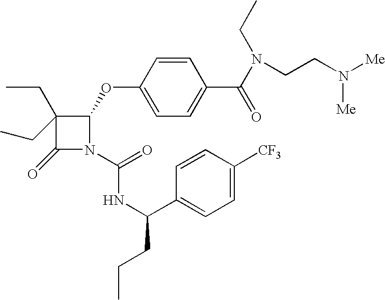

### 2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide

### Step A: Preparation of 2-(S)-((4-(benzyloxy)carbonyl)phenoxy)-3,3-diethyl-N-(1-(R)-(4-(trifluoromethyl)phenyl)but-3-en-yl)-4-oxo-1-azetidinecarboxamide

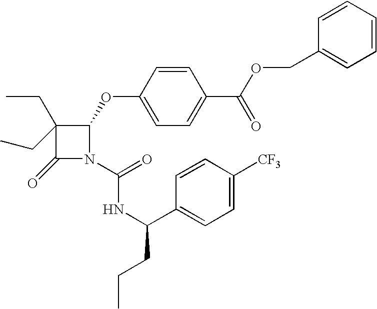

Using the procedure of Example 1, Step A; a toluene solution of (R)-α-allyl-4-(trifluoromethyl)benzyl isocyanate (Intermediate 3, from 1.5 gm of N—((R)-α-allyl-4-(trifluoromethoxy)benzyl)-(S)-(−)-t-butylsulfinamide) and (S)-2-[4-(benzyloxycarbonyl)phenoxy]-3,3-diethyl-azetidin-4-one (Intermediate 1 from above) (0.80 gm), the title product (1.22 gm) was obtained.

### Step B: Preparation of 2-(S)-(4-(carboxy)phenoxy)-3,3-diethyl-N-[1-(R)-(4-(trifluoromethyl)phenyl)butyl]-4-oxo-1-azetidinecarboxamide

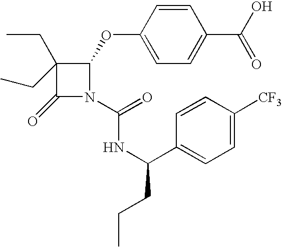

Using the procedure of Example 1, Step B, 2-(S)-(4-(benzyloxycarbonyl)phenoxy)-3,3-diethyl-N-(1-(R)-(4-(trifluoromethyl)phenyl)but-3-en-yl)-4-oxo-1-azetidinecarboxamide (1.20 gm) (from Example 2, Step A) afforded the title compound (1.0 gm) as a white foam.

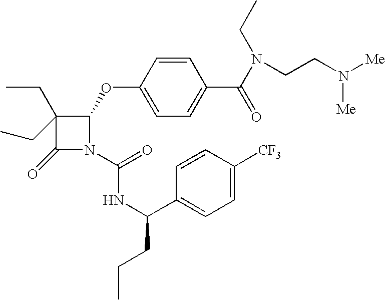

### Step C: Preparation of 2-(S)-[4-(((2-(dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide

Using the procedure of Example 1, Step C, 2-(S)-(4-(carboxy)phenoxy)-3,3-diethyl-N-[1-(R)-(4-(trifluoromethyl)phenyl)butyl]-4-oxo-1-azetidinecarboxamide (1.0 gm) (from Example 2, Step B) afforded the title product as a partially white solid (1.03 gm).

NMR (CD3CN): δ 0.900 and 0.904 (2 t, 6H), 1.01 (t, 3H), 1.0-1.2 (br m, 3H), 1.2-1.4 (m, 2H), 1.7-1.9 (2 m, 5H), 1.9-2.3 (m, 7H), 2.3-2.5 (br m, 2H), 3.2-3.4 (2 br m, 4H), 4.80 (q, 1H), 5.73 (s, 1H), 6.92 (d, 1H), 7.18 (dt, 2H), 7.25 (br d, 2H), 7.30 (dt, 2H), 7.39 (dt, 2H).

HPLC/MS (reverse phase, 10-98% CH₃CN/0.05% TFA in water/0.05% TFA over 5.5 min): Rt=3.43 min, m/e=605 (M+1, 100%).

### Example 4

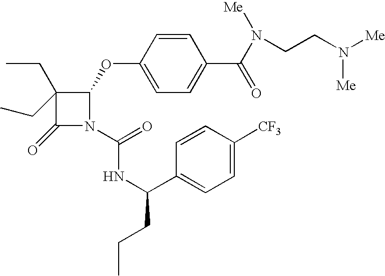

### 2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide

Using the procedure of Example 3, Step C, but using N,N,N-trimethylethylenediamine, 2-(S)-(4-(carboxy)phenoxy)-3,3-diethyl-N-[1-(R)-(4-(trifluoromethyl)phenyl)butyl]-4-oxo-1-azetidinecarboxamide (31 mg) (prepared as in Example 2, Step B) afforded the title product (33 mg).

HPLC/MS (reverse phase, 10-98% CH₃CN/0.05% TFA in water/0.05% TFA over 5.5 min): Rt=3.70 min, m/e=591 (M+1, 100%).

### Example 5

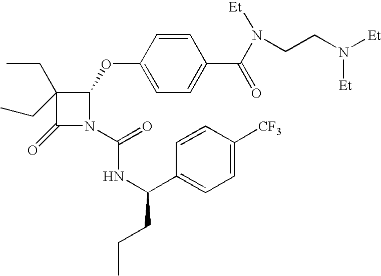

### 2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide

Using the procedure of Example 3, Step C, but using N,N,N-triethylethylenediamine, 2-(S)-(4-(carboxy)phenoxy)-3,3-diethyl-N-[1-(R)-(4-(trifluoromethyl)phenyl)butyl]-4-oxo-1-azetidinecarboxamide (31 mg) (prepared as in Example 2, Step B) afforded the title product (33 mg).

HPLC/MS (reverse phase, 10-98% CH₃CN/0.05% TFA in water/0.05% TFA over 5.5 min): Rt=3.88 min, m/e=633 (M+1, 100%).

### Example 6

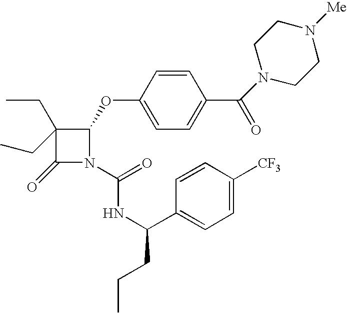

### 2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide

Using the procedure of Example 3, Step C, but using 4-methylpiperidine, 2-(S)-(4-(carboxy)phenoxy)-3,3-diethyl-N-[1-(R)-(4-(trifluoromethyl)phenyl)butyl]-4-oxo-1-azetidinecarboxamide (32.5 mg) (prepared as in Example 2, Step B) afforded the title product (32 mg).

HPLC/MS (reverse phase, 10-98% CH₃CN/0.05% TFA in water/0.05% TFA over 5.5 min): Rt=3.59 min, mile=589 (M+1, 100%).

### Biological Data for Examples 1-6

As exemplified in Table 1, the Examples 1-6 of this application were determined to be potent, mechanism-based inactivators of Human Leukocyte Elastase (HLE) in two different in vitro time-dependent incubation assays. The two assays are described at the end of this Biological Data Section in Note 1. See the literature references in Note 2 for a discussion of the mechanism for this type of beta-lactam inactivation of HLE. Two sources of enzyme were used; either purified elastase or crude extracts of human polymorphonuclear leukocytes (PMN) containing elastase activity. In the assay utilizing purified HLE, K_{inact}/Kᵢ values between 290,000-827,000 M⁻¹ sec⁻¹ were observed. In the second analogous assay utilizing the PMN cell lysate source of HLE, slightly higher K_{inact}/Kᵢ values between 375,000-1,320,000 M⁻¹ sec⁻¹ were observed. For the compound of Example 1, the results were 638,000 M⁻¹ sec⁻¹ (average of n=3) and 809,000 M⁻¹ sec⁻¹ (n=4), respectively, and for Example 3, 804,000 M⁻¹ sec⁻¹ (average of n=4) and 952,000 M⁻¹ sec⁻¹ (n=5). The inactivation rate for the comparator example from U.S. Pat. No. 5,591,737 (see Patent Table 9, Compound #290) was determined under the same, current purified HLE assay conditions to be 523,000 M⁻¹ sec⁻¹. Note that in this assay, head-to-head comparisons are particularly valuable. For example, the current purified HLE assay result for Compound #290 was about one-half the value previously obtained in a similar inactivation assay which utilized a different substrate and afforded a K_{observed}/I value of 1,010,000 M⁻¹ sec⁻¹ as reported for Compound #290 in U.S. Pat. No. 5,591,737 (see Notes 2a and 3).

An important and desirable aspect of these mechanism-based HLE inhibitors is also the extent to which the enzyme remains inactivated or, in other words, the rate at which the modified inhibitor comes off the enzyme after the inactivation event has occurred and which then gives back the active HLE enzyme (see Note 2c and 4). This property was assessed in a time-dependent reactivation assay and the results were calculated as a first-order rate constant expressed as their half-lives in hours (t_{1/2}). The first-order off-rate constant for Examples 1 and 3 of this application were determined to be 17.4 and 18.4 hrs (n=3), respectively. Under head-to-head conditions (n=2) with Examples 1 and 3, the off-rate for the comparator Compound #290 of U.S. Pat. No. 5,591,737 was determined to be 7.1 hrs, or about 2.5-2.6 times as fast.

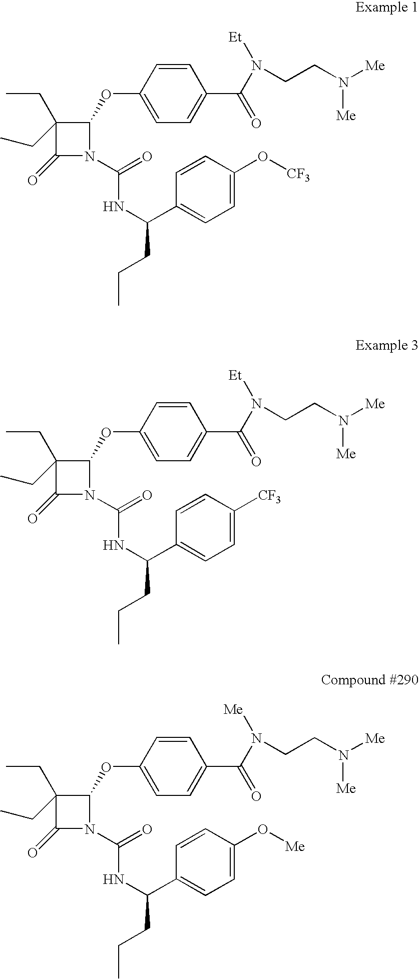

TABLE 1
HLE Inhibition Data

| | K_{inact}/Kᵢ (M⁻¹sec⁻¹) | K_{inact}/Kᵢ (M⁻¹sec⁻¹) | Off-Rate |
|---|---|---|---|
| Compound | Purified HLE | Lysate assay | t_{1/2} (hrs) |
| Example 1 | 638,000 (n = 3) | 809,000 (n = 4) | 17.4 hrs (n = 3) |
| Example 2 | nd | 738,000 (n = 2) | nd |
| Example 3 | 804,000 (n = 4) | 952,000 (n = 5) | 18.4 hrs (n = 3) |
| Example 4 | 290,000 (n = 1) | 427,000 (n = 2) | nd |
| Example 5 | nd | 375,000 (n = 2) | nd |
| Example 6 | 827,000 (n = 3) | 1,320,000 (n = 3) | nd |
| Compound | 523,000 (n = 3) | nd | 7.1 hrs (n = 2) |
| #290* | | | |

*U.S. Pat. No. 5,591,737; Table 9, Compound #290; reported K_{observed}/I = 1,010,000 M⁻¹sec⁻¹
(Note 2a)

As exemplified in Table 2, the in vitro human liver microsome preparation stability results (see Note 5 for a literature reference protocol) for Examples 1-6 of this application were determined in a head-to-head experiment (Assay #1) and are expressed as the first-order rate constants (t_{1/2}) in minutes for the disappearance of substrate and the amount of substrate left after a 30 minute incubation at 37° C. The results for Examples 1 and 3 were t₁₁₂=87 and 32 minutes with 78% and 60% remaining after 30 minutes, respectively, compared to shorter half-lives and lesser amounts left for Examples 2 and 4-6 (see Note 6 for conditions).

Under similar in vitro human liver microsome preparation stability assay conditions at 37° C. for 30 or 60 minutes in a separate head-to-head experiments (Assay #2 and #3) (see Note 6 for conditions) with Examples 1 and 3 and the comparator Compound #290 from U.S. Pat. No. 5,591,737, the amounts left after the 30 minute incubation (with 0.05 mg/mL microsome protein vs 0.1 mg/mL for Assay #3) were 67, 99 and 79% and for the 60 minute incubation were 94, 73 and 77%, respectively. Similarly, in a third repeat assay (Assay #4 with—NADPH controls) (see Note 6 for conditions), 90 and 72% were remaining for Example 1 and the comparator compound, respectively. Overall, the in vitro human liver microsome stabilities for Examples 1 and 3 were at least comparable to, or better than, that for Compound #290. These results might indicate an in vivo rate of liver metabolism of Example 1 and 3 similar to, or better than, the comparator Compound #290 in man.

TABLE 2
Human Liver Microsome Stability Data
Assay #1

| | t_{1/2} | % Left |
|---|---|---|
| Compound | (min) | @ 30 min |
| Example 1 | 87 | 78% |
| Example 2 | 12 | 21% |
| Example 3 | 32 | 60% |
| Example 4 | 25 | 51% |
| Example 5 | 30 | 31% |
| Example 6 | 12 | 12% |

Assay #2 and #3

| | % Left Assay 2 | % Left Assay 3 |
|---|---|---|
| Compound | @ 0, 2.5, 5, 10, 15, 30 min | @ 0, 5, 10, 15, 30 60 min |
| Example 1 | 100, 78, 78, 73, 70, 67 +/− 2 | 100, 101, 79, 90, 100, 94 +/− 10% |
| Example 3 | 100, 107, 85, 82, 82, 79 +/− 7% | 100, 86, 81, 75, 75, 73 +/− 0% |
| Compound #290* | 100, 99, 95, 89, 82, 79 +/− 6% | 100, 90, 88, 85, 83, 77 +/− 4% |

Assay #4

| | % Left (+NADPH) | % Left (−NADPH) |
|---|---|---|
| Compound | @ 0, 5, 10, 15, 30, 60 min | @ 0 and 60 min |
| Example 1 | 100, 104, 99, 96, 91, 90 +/− 4% | 87 +/− 21 and 99 +/− 2% |
| Compound #290* | 100, 92, 89, 84, 78, 72 +/− 0% | 89 +/− 5 and 83 +/− 1% |

*U.S. Pat. No. 5,591,737; Table 9, #290

In a separate experiment as exemplified in Table 3, the stability of the current Example 1 and the comparator Compound #290 in incubations with microsome preparations from 4 different species were also determined (Note 5). Overall, the human, dog, rat and monkey microsome stabilities of Example 1 were at least comparable to, or better than, those for Compound #290. These results predict an in vivo rate of metabolism of Example 1 in man similar to that observed in the three commonly used pre-clinical species rat, dog and monkey and that the metabolism of Example 1 would be similar to, or better than, that of the comparator Compound #290 in all four species.

TABLE 3
Liver Microsome Stability Data in 4 Species*

| | | % Left | % Left |
|---|---|---|---|
| Compound | Species | @ 30 min | @ 60 min |
| Example 1 | Human | 92 | 92 |
| | Dog | 93 | 86 |
| | Rat | 96 | 94 |
| | Monkey | 91 | 77 |
| Example 3 | nd | | |
| Compound #290** | Human | 93 | 88 |
| | Dog | 100 | 102 |
| | Rat | 99 | 93 |
| | Monkey | 90 | 67 |

*Substrate concentration = 0.5 uM; Microsome concentration = 0.25 mg/mL
**U.S. Pat. No. 5,591,737; Table 9, #290

As exemplified in Table 4, the inhibition of three representative CYP-P450 isoforms were evaluated for Example 1 and the comparator Compound #290 and were found to be very moderate, being above 8 μM in all cases (Note 7). As exemplified in Table 5, there was also minimal time-dependent inhibition for the CPY-3A4 isoform for both Examples 1 and 3 and the comparator Compound #290, even at the higher 50 μM concentration (rate=0.018 min⁻¹ for both Example 1 and Compound #290, compared to 0.006 min⁻¹ for solvent and 0.048 min⁻¹ for the TAO positive control) (Note 8). In addition, as exemplified in Table 6, both Examples 1 and 3 and the comparator Compound #290 showed minimal effects (5%, 0% and 6%, respectively) compared to the Rifampicin standard (defined as 100% at 10 μM) in an assay of CYP-3A4 induction (Note 9). Since there were no significant differences between Examples 1 and 3 versus Compound #290 in any of these three CYP-P450 assays, the likely drug-drug interactions in the clinic would be expected to be similar at comparable exposure levels.

TABLE 4
CYP-P450 Reversible Inhibition

| | IC₅₀ (μM) | | |
|---|---|---|---|
| Compound | 3A4 | 2C9 | 2D6 |
| Example 1 | 46 | 17 | 37 |
| Example 3 | 39 | 8.1 | 41 |
| Compound #290* | 62 | 25 | 39 |

*U.S. Pat. No. 5,591,737; Table 9, #290

TABLE 5
P450 CYP-3A4 Time-Dependent Inhibition

| Compound | @ 10 μM (#1) | @ 10 μM (#2) | @ 50 μM |
|---|---|---|---|
| Example 1 | 0.005 min⁻¹ | 0.008 min⁻¹ | 0.018 min⁻¹ |
| Example 3 | 0.014 | nd | nd |
| Compound #290* | 0.017 min⁻¹ | 0.008 min⁻¹ | 0.018 min⁻¹ |
| Solvent | 0.008 | 0.006 min⁻¹ | 0.006 min⁻¹ |
| TOA | 0.050 | 0.040 min⁻¹ | 0.048 min⁻¹ |

*U.S. Pat. No. 5,591,737; Table 9, #290

TABLE 6
P450 CYP-3A4 Induction

| | CYP-3A4 Induction |
|---|---|
| Compound | % of Rifampicin response @ 10 μM |
| Example 1 | 5% |
| Example 3 | 0% |
| Compound #290* | 6% |

*U.S. Pat. No. 5,591,737; Table 9, #290

As exemplified in Table 7, the in vivo pharmacokinetic profiles for Examples 1 and 3 and the comparator Compound #290 were determined in the rat at the standard screening doses of 1 mpk i.v. and 2 mpk p.o. (Note 10). While the oral bioavailability appeared to be comparable (F=25%, 28% and 29%, respectively), examination of the i.v. clearance rates (Cl=42, 113 and 114 mL/min/kg, respectively) and plasma exposure levels (i.v. AUC_{N}=0.65, 0.24 and 0.28 μMhrkg/mg; p.o. AUC_{N}=0.16, 0.069 and 0.081 μMhrkg/mg, respectively) indicated that Compound #290 would be expected to require at least a 2-fold higher dose to maintain the same plasma drug exposure level as Example 1 in a clinical setting, while Example 3 had about the same exposure as Compound #290.

TABLE 7
Rat Pharmacokinetics

| Compound | Example 1 | Example 2 | Compound #290* |
|---|---|---|---|
| i.v. dosing @ 1 mpk | | | |
| AUC_{N} (μMhrkg/mg) | 0.65 | 0.24 | 0.28 |
| Cl (mL/min/kg) | 42 | 113 | 114 |
| VDₛₛ (L/kg) | 11 | 29 | 14.5 |
| t_{1/2} | 3.1 hr | 3.7 | 2.0 hr |
| p.o. dosing @ 2 mpk | | | |
| AUC_{N} (μMhrkg/mg) | 0.16 | 0.069 | 0.081 |
| F | 25% | 28% | 29% |

*U.S. Pat. No. 5,591,737; Table 9, #290

The observations summarized in Table 8 indicate that both Example 1 and Compound #290 have the same NO Effect Level in a Dose Limiting Toxicity assay in the rat (NOEL=100 mpk/day for 7 days for both) (Note 11), and would argue against the use of higher doses of Compound #290 to achieve the same plasma exposure as Example 1 in the clinic.

TABLE 8
Rat 7 Day Oral Dose Limiting Toxicity Assessment

| | 25 mpk | 100 mpk | 750 mpk |
|---|---|---|---|
| xample 1 | No effects | No effects | Significant toxicity observed |
| Example 2 | not done | not done | not done |
| Compound #290* | No effects | No effects | Significant toxicity observed |

*U.S. Pat. No. 5,591,737; Table 9, #290

In summary, a comparative efficacy ratio for Examples 1 and 3 versus the comparator Compound #290 can be derived from the product of the ratios of the inactivation rates (K_{inact}/Kᵢ), times the ratio of the reactivation off-rates times (t_{1/2}), times the ratio of the normalized p.o. plasma exposures for Example 1 divided by Compound #290 or Example 3 divided by Compound #290. As calculated below, the above results predict an efficacy ratio 6 times better for Example 1 compared to the comparator Compound #290 from U.S. Pat. No. 5,591,737 (Table 9) and a ratio of 3.3 for Example 3. The fact that the drug-drug interaction profile based on the minimal CYP-P450 effects for both Example 1 and 3 as well as Compound #290 and the comparable rat Dose Limiting Toxicity results for Example 1 and Compound #290 would argue against the possible ability to use a higher dose of Compound #290 vs the exemplified most preferred Examples 1 and 3.

### Efficacy Ratio for Example 1:

$$Efficacy ratio Example 1 = \frac{( \frac{Example 1 {K}_{inact}}{{K}_{i}} )}{( \frac{Compound \#290 {K}_{inact}}{{K}_{i}} )} \times \frac{( Example 1 off \text{-} rate {t}_{12} )}{( Compound \#290 off \text{-} rate {t}_{1 / 2} )} \times \frac{( Example 1 p . o . {AUC}_{N} )}{( Compound \#290 p . o . {AUC}_{N} )}$$

$$Efficacy ratio Example 1 = ( \frac{638 , 0000 {M}^{- 1} {sec}^{- 1}}{523 , 000 {M}^{- 1} {sec}^{- 1}} ) \times ( \frac{17.4 hrs}{7.1 hrs} ) \times ( \frac{0.16 μMhrkg \text{/} mg}{0.081 μMhrkg \text{/} mg} )$$

$$Efficacy ratio Example 1 = 1.2 \times 2.5 \times 2.0 \\ Efficacy ratio Example 1 = 6.0$$

### Efficacy Ratio for Example 3:

$$Efficacy ratio Example 3 = \frac{( \frac{Example 3 {K}_{inact}}{{K}_{i}} )}{( \frac{Compound \#290 {K}_{inact}}{{K}_{i}} )} \times \frac{( Example 3 off \text{-} rate {t}_{1 / 2} )}{( Compound \#290 off \text{-} rate {t}_{1 / 2} )} \times \frac{( Example 3 {AUC}_{N } )}{( Compound \#290 {AUC}_{N} )}$$

$$Efficacy ratio Example 3 = ( \frac{804 , 000 {M}^{- 1} {sec}^{- 1}}{523 , 000 {M}^{- 1} {sec}^{- 1}} ) \times ( \frac{18.4 hrs}{7.1 hrs} ) \times ( \frac{0.069 μMhrkg \text{/} mg}{0.081 μMhrkg \text{/} mg} )$$

$$Efficacy ratio Example 3 = 1.5 \times 2.6 \times 0.85 \\ Efficacy ratio Example 3 = 3.3 .$$

### Notes for Tables 1-8.

### Note 1: Enzyme Assays for the Inhibition of Human Polymorphonuclear Leukocyte Elastase Via Hydrolysis of N-Methyoxysuccinyl-alanyl-alanyl-prolylalanine-p-nitroanilide Reagent.

Prepare substrate by first dissolving in DMSO to 30 mg/ml (50 mM). Further dilute the substrate to 10 mM and add 25 μl to a final volume of 250 μl for a final concentration of 1 mM in the assay.

Prepare the enzyme in TES Buffer (0.045M TES (N-tris[hydroxymethyl]methyl-2-mino-ethanesulfonic acid), 0.45 M NaCl, pH 7.5.). Two sources of enzyme were used; either crude extracts of human polymorphonuclear leukocytes (PMN) containing elastase activity or purified elastase. The PMN lysates were prepared by isolating human neutrophils from whole blood after dextran sedimentation, ficoll density separation and red blood cell lysis. The neutrophils were lysed in PBS with sonciation and the concentration of active enzyme was determined by utilizing a standard curve of purified enzyme. For the purified elastase, the enzyme was resuspended at 10 mg/ml (˜340 μM) in 0.02M NaOAc, 50% glycerol. For both the PMN lysate and purified enzyme, a further dilution of enzyme was made to 30 nM and 25 μl was added to a final volume of 250 μl. The final concentration of enzyme in the assay was 3 nM.

The inhibitors to be tested were dissolved in DMSO at 10 mM. Directly before use, the inhibitors were further diluted into TES Buffer to 265 nM and 25 μA was added to a final volume of 250 μl. The final concentration of inhibitor in the assay was 26.5 nM.

In a 96-well plate (flat bottom, polystyrene) 175 μl TES Buffer, 25 μl substrate, 25 μl inhibitor and 25 μl enzyme were added. After thoroughly mixing, the plate was then read in a spectrophotometer at 410 nm for 30 minutes counting every 10 seconds. Results were expressed as K_{inact}/Kᵢ which is the second order rate constant in per mole per second for inactivation of the enzyme.

### Note 2: HLE Inactivation Assay and Mechanism Reference.

(a) J. B. Doherty, et. al., U.S. Pat. No. 5,591,737, column 21.

(b) W. B. Knight, et. al., Biochemistry, 1992, 31, 8160.

(c) B. G. Green, et. al., Biochemistry, 1995, 34, 14331.

### Note 3: K_{inact}/Kᵢ Formula.

K_{inact}/Kᵢ=K_{obs}/[I]*(1+[S]/Kₘ); where K_{obs} is calculated by GraphPad Prism by the equation Y=[vs+(v0−vs)*(1−exp(−k_{obs}*x))]/k_{obs}+Y0; and I=26.5 nM, S=1 mM and Kₘ=0.14 mM.

### Note 4: HLE Off-Rate Protocol.

Elastase was incubated with 100-fold molar excess of inhibitor in TES buffer (0.045M TES, 0.45M NaCl, pH 7.5, 0.1% BSA) at room temperature for 60 minutes. After the incubation was complete, the samples were loaded onto prepared columns (Micro BioSpin Chromatography Columns-Bio-Gel P-6 Columns Bio-Rad Cat# 732-6221; prepared as directed by the manufacturer). The columns were centrifuged for 4 minutes at 1000 g. The filtrate was collected and maintained at 37° C. At specific time points, the filtrate was monitored for elastase activity by adding the filtrate to 1 mM substrate (MeoSucc-AAPV-pNA; Elastin Products Cat# FH237) in TES buffer (0.045M TES, 0.45M NaCl, pH 7.5) and measuring the absorbance at 410 nm. The absorbance was monitored every 10 seconds for 5 minutes. The difference in the rates of pNA formation in the absence of compound was used to calculate the % bound in the presence of compound.

See Note 2c reference for a similar assay description and discussion of the off-rate for related beta-lactam time-dependent HLE inhibitors.

### Note 5: Microsome Assay Protocol Reference.

X. S. Tong, et. al., J. of Chromatography, 2006, 833, 165.

Note 6: Human Liver Microsome Assay # 1-3 conditions.
Assay experiment #1: Substrate concentration=1 μM; Protein concentration=0.1 mg/mL; incubation time=30 minutes at 37° C.
Assay experiment #2: Substrate concentration=1 μM; Protein concentration 0.1 mg/mL; incubation time=30 minutes at 37° C.
Assay experiment #3: Substrate concentration=1 μM; Protein concentration=0.1 mg/mL; incubation time=60 minutes at 37° C.
Assay experiment #4: Substrate concentration=1 μM; Protein concentration=1.0 mg/mL; incubation time=60 minutes at 37° C.

### Note 7: CYP-P450 Reversible Inhibition Assay Protocol.

This assay measured the reversible inhibition of the test compounds against three cytochrome P450 isozymes (CYP-P450 3A4, 2C9 and 2D6) in an automated screening formate. Inhibition of the P450 activity in human liver microsomes or recombinant P450 microsomes was measured using a probe which upon metabolism, was converted to a metabolite which exhibits fluorescent properties at specific excitation and emission wavelengths. Cumene hydroperoxide, NADPH or NADPH regenerating systems can be used as electron donors for the P450 catalytic cycle. Control incubations containing no inhibitor or no fluorescent probe were performed to evaluate the 100% and 0% activities, respectively. The activity of P450 enzyme was evaluated in the presence of 0.137, 0.273, 0.547, 1.094, 2.188, 4.375, 8.750, 17.5, 35.0 and 70.0 μM concentrations of the test compounds. Inhibition curves were generated and IC₅₀ values were calculated for each tested compound using standard techniques.

### Note 8: P450 CYP-3A4 Time-Dependent Inhibition Assay Protocol.

This assay measured the pre-incubation time-dependent inhibition of potential CYP-3A4 inhibitors in human liver microsomes using the CYP-3A4 mediated testosterone 6-beta-hydroxylation formate in the presence of test compounds in a semi-automated screening using LC-MS/MS detection. Test compounds are pre-incubated at 37° C. with human liver microsomes (2.5 mg/mL protein) and a NADPH regenerating system for 10, 20, 30 and 40 minutes prior to a ten-fold dilution with a 250 μM solution of testosterone. The diluted reaction is then incubated for 15 minutes at 37° C. The concentration of 6-β-hydroxytestosterone metabolite formed is measured by LC-MS/MS using validated analytical methods. The percent activity remaining is calculated by comparing the amount of marker metabolite formed in the presence of inhibitor compared to no inhibitor control samples at corresponding time points. No inhibitor control samples represent the maximum amount of 6-β-hydroxytestosterone formed under these assay conditions.

### Note 9: P450 CYP-3A4 Induction Assay Protocol.

This assay measured the in vitro induction of the human CYP-3A4 enzyme in the presence of 0.37, 1.11, 3.33, 10 and 30 μM test compound in an automated formate. The results are expressed as the relative percent induction measured for test compound at the 10 μM concentration compared to the known inducer standard Rifampicin at 10 μM (defined as 100%). (See Durocher, et. al., Anal. Biochem. 2000, 284, 316 for details of the assay).

### Note 10: PK Protocol.

Compounds were formulated as their mono-L-malic acid salts at 1 mg/mL in PEG-200:water 70:30 (v:v) and administered to Sprague-Dawley rats (n=2 for i.v. and n=3 for p.o. arms) at a dose of 1 mg/kg i.v. or 2 mgk p.o. (free base equivalent). Blood samples were taken at 0.03, 0.08, 0.25, 0.50, 1, 2, 4, 6, 8 and 24 hours for the i.v. arm and at 0.25, 0.50, 1, 2, 4, 6, 8 and 24 hours for the p.o. arm. The plasma concentrations of test compound at each time point were determined by protein precipitation with acetonitrile followed by liquid chromatography—tandem mass spectrometry versus a standard curve. The PK parameters were calculated using validated techniques known in the literature (see Stedman's Medical Dictionary, 23rd ed.).

### Note 11: Rat Dose-Limiting Toxicity Protocol.

Example 1 and Compound #290 as their mono-L-malic acid salts were formulated in 0.5% Methocel and dosed p.o. at 25, 100 and 750 mg per k per day (free base equivalent) for 7 days (n=5 animals in each group). During the dosing period observation of any physical signs of toxicity were noted. Upon termination on day 8, a standard battery of blood chemistry and tissue analyses were performed and evaluated for significant effects compared to vehicle controls. Other than salivation seen for both Example 1 and Compound #290 at the 100 mpk dose, no adverse effects were observed at the 25 and 100 mpk doses. At the 750 mpk dose level several adverse effects were observed including mortality (⅖ for Example 1 on Day 7 and 8 in the 750 mpk dose group; ⅕ on day 4 for Compound #290 in the 750 mpk dose group).

For treatment as described above the compounds of the invention may be administered orally, topically, parenterally, by inhalation spray or rectally in dosage unit Formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, dogs, cats, etc., the compounds of the invention are effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The said aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives.

Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan mono-oleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution glucose in water and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula (I) may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the anti-inflammatory agents are employed.

The amount of active ingredient(s) that may be combined with the carrier materials to produce a single dosage form will vary depending upon the host treated and the particular mode of administration.

For treatment as described above the compounds of Formula (I) may be administered orally, topically, parenterally, by inhalation spray or rectally in dosage unit Formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, dogs, cats, etc., the compounds of the invention are effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The said aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives.

Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan mono-oleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution glucose in water and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the anti-inflammatory agents are employed.

The amount of active ingredient(s) that may be combined with the carrier materials to produce a single dosage form will vary depending upon the host treated and the particular mode of administration.

For example, a formulation intended for the oral administration of humans may contain from 5 mg to 500 mg of each active agent(s) compounded with an appropriate and convenient amount of carrier material which may vary from about 5 to about 95 percent of the total composition. For purposes of this specification, this broad dosage range is specifically intended to include, but is not limited to, range of 5 mg to 500 mg; 5 mg to 250 mg; 10 mg to 500 mg; 10 mg to 250 mg; 25 mg to 500 mg; and 25 mg to 250 mg. It is further anticipated that the adult may be administered up to 500 mg of elastase inhibitor per day. This daily dosage may be divided into 2 or three doses per day. Examples of such doses include, 2.5, 5, 10, 12.5, 25, 50, 100, 125, and 250 mg administered twice a day.

Furthermore, it is also possible that most effective treatment may warrant administration of an initial dosage of one range (e.g. 250 mg or 500 mg in a dose) followed by administration of a second (lower) range (e.g. 2.5, 5, 10, 12.5, 25, 50, 100, 125) twice a day.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

What is claimed is:

1. A compound selected from the group consisting of
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof.

2. A compound of claim 1 which is
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide, or a pharmaceutically acceptable salt thereof.

3. A compound of claim 1 which is
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide, or a pharmaceutically acceptable salt thereof.

4. A pharmaceutical composition comprising a compound selected from
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-di ethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-di ethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof, and a pharmaceutically acceptable carrier.

5. A method of treating a disease selected from the group consisting of emphysema, bronchial inflammation, chronic bronchitis, cystic fibrosis, and acute respiratory distress syndrome in a patient having said disease comprising the administration of a non-toxic therapeutically effective amount of a compound selected from:
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof.

6. A method of treating a disease selected from the group consisting of rheumatoid arthritis and osteoarthritis in a patient having said disease comprising the administration of a non-toxic therapeutically effective amount of a compound selected from
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof.

7. A method of treating a disease selected from the group consisting of glomerulonephritis, spondylitis, lupus, psoriasis, atherosclerosis, sepsis, septicemia, shock, myocardial infarction, reperfusion injury, and periodontitis in a patient in need of such treatment comprising the administration therapeutically effective amount of a compound selected from
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof.

8. A method of treating a disease selected from idiopathic mylofibrosis, polycythemia vera, essential thrombocytopenia, aortic aneurism, advanced coronary artery disease and pulmonary hypertension in a patient in need of such treatment comprising the administration therapeutically effective amount of a compound selected from
2-(S)-[4-(((2-(Dimethylamino)ethyl)ethylamino)carbonyl)phenoxy]-3,3-diethyl-N-[1-(R)-(4-(trifluoromethoxy)phenyl)butyl]-4-oxo-1-azetidinecarboxamide,
2-(S)-[4-((4-methylpiperidin-1-yl)carbonyl)phenoxy]-3,3-diethyl-N-[1-(R)-(4-(trifluoromethoxy)phenyl)butyl]-4-oxo-1-azetidinecarboxamide,
2-(S)-[4-(((2-(Dimethylamino)ethyl)ethylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Dimethylamino)ethyl)methylamino)carbonyl)phenoxy]-3,3-diethyl-N-[1-(R)-(4-(trifluoromethyl)phenyl)butyl]-4-oxo-1-azetidinecarboxamide,
2-(S)-[4-(((2-(Diethylamino)ethyl)ethylamino)carbonyl)phenoxy]-3,3-diethyl-N-[1-(R)-(4-(trifluoromethyl)phenyl)butyl]-4-oxo-1-azetidinecarboxamide, and
2-(S)-[4-((4-methylpiperidin-1-yl)carbonyl)phenoxy]-3,3-diethyl-N-[1-(R)-(4-(trifluoromethyl)phenyl)butyl]-4-oxo-1-azetidinecarboxamide
or a pharmaceutically acceptable salt thereof.

9. Use of a compound of claim 1 in the treatment of a disease selected from idiopathic mylofibrosis, polycythemia vera, essential thrombocytopenia, aortic aneurism, advanced coronary artery disease and pulmonary hypertension.

10. Use of a compound of claim 1 in the treatment of a disease selected from emphysema, bronchial inflammation, chronic bronchitis, cystic fibrosis, acute respiratory distress syndrome, rheumatoid arthritis, osteoarthritis; glomerulonephritis, spondylitis, lupus, psoriasis, atherosclerosis, sepsis, septicemia, shock, myocardial infarction, reperfusion injury, and periodontitis.
